(19) United States
(12) Patent Application Publication
(10) Pub. No.: US 2015/0168391 A1
(43) Pub. Date: Jun. 18, 2015

(54) METHOD FOR TREATING A POROUS MEMBRANE AND USES THEREOF

(71) Applicant: Agency for Science, Technology and Research, Singapore (SG)

(72) Inventors: Jackie Y. Ying, Singapore (SG); Yong Yeow Lee, Singapore (SG); Jianhao Bai, Singapore (SG)

(21) Appl. No.: 14/400,472

(22) PCT Filed: May 13, 2013
(86) PCT No.: PCT/SG2013/000190

§ 371 Date: Nov. 11, 2014

(30) Foreign Application Priority Data
May 11, 2012 (SG) 201203480-7

(51) Int. Cl. G01N 33/543

(52) U.S. Cl. CPC G01N 33/54366

(57) ABSTRACT

The present invention relates to a method for treating a porous membrane, said method comprising contacting said porous membrane with at least one alcohol to reduce the pore size of said porous membrane relative to an untreated porous membrane. The invention further relates to an apparatus comprising the treated porous membrane.

### FIELD OF INVENTION

The present invention relates to the treatment of a porous membrane and the use of the treated membrane as an immunoassay platform.

### BACKGROUND

One of the most cost-effective immunoassay platforms for point-of-care (POC) diagnostic kits remains the lateral flow platform. This technology has allowed an untrained individual to perform qualitative health screening tests (e.g. infectious diseases, women's health issues, and cardiovascular diseases) on site, without the need to accumulate sufficient samples or run immunoassays that are comparable to diagnostic laboratories.

A typical lateral flow platform involves the use of a porous membrane to transport biological samples through a strip via capillary forces. This allows for simplicity in design and negates the need for pumps, valves and other electronic parts.

However, most commercial lateral flow POC diagnostic kits tend to be qualitative instead of quantitative. Enzyme-linked immunosorbent assays (ELISA), on the other hand, provide an ideal benchmark for performing quantitative assays of desired analytes within samples. ELISA are typically performed in 96 or up to 384 wells. However, the relatively large sample volume (typically from 20 μL-100 μL) required per well and the need for a calibration curve render ELISA technology challenging for POC diagnostic kits.

Accordingly, there has been an increasing interest for multiplexed ELISA analysis requiring low sample volumes for potential applications as POC diagnostic kits. This was recently demonstrated by providing an array of probes on a polydimethylsiloxane (PDMS) surface, and the implementation of fluorescence ELISA, which allows for the miniaturization of assays without losing sensitivity. However, the results of such assays require a barcode reader for analysis, resulting in additional cost for the end users which may be prohibitive.

Compared to fluorescence ELISA, colorimetric ELISA is more cost-effective and practical. A known colorimetric ELISA technology utilizes porous membranes, termed as paper-ELISA (p-ELISA), to allow for low-volume sample analysis via ELISA technology. In one known p-ELISA technology, segmented patterns (spanning the entire membrane thickness) are created within the porous membranes to generate “wells” suitable for ELISA applications. Such patterns can be created through the use of photoresist, PDMS, wax printing, paper sizing and screen printing.

It has been demonstrated in known p-ELISA applications that only about 3 μL of sample per well was required to generate sufficient colorimetric output for visual observation and analysis. Like conventional lateral flow kits, p-ELISA is simple in design and does not require pumps, valves and other electronic parts.

However, a problem with paper-ELISA is the difficulty in obtaining “wells” with high aspect ratios due to wicking of the sample reagents across the porous membranes. Specifically, wicking results in “wells” having a large width feature and may cause mixing of contents between wells disposed adjacent to each other. This drawback limits the ability of paper-ELISA to provide high resolution “wells”, wherein a high density of sample wells are located in close proximity to each other without their contents mixing.

This drawback also results in a need to create segmented patterns spanning the thickness of the porous membrane to ensure that the contents of one well are spatially separated from the contents of an adjacent well. However, this in turn makes it difficult to wash the porous membrane after performing the assay.

Accordingly, there is a need to provide a platform for p-ELISA technology that overcomes or at least ameliorates the disadvantages described above. In particular, there is a need to provide a porous membrane for use in p-ELISA that is capable of providing high resolution wells without the need for distinct segmentation of the porous membrane.

### SUMMARY

In one aspect, there is provided a method for treating a porous membrane, said method comprising contacting said porous membrane with at least one alcohol to reduce the pore size of said porous membrane relative to an untreated porous membrane.

In an embodiment, there is provided a method for preparing a porous membrane having closely packed data points for membrane-based colorimetric ELISA capable of semi-quantitative analysis, the method comprising a step of contacting said porous membrane with at least one alcohol to reduce the pore size of said porous membrane relative to an untreated porous membrane.

Surprisingly, the inventors have found that the wicking rate of reagents is advantageously reduced when dispensed onto a membrane that has been treated with at least one alcohol. Without being bound by theory, it is postulated that this could be due to the displacement of air within the porous membrane by the low surface tension alcohol during the treatment process. Subsequent removal of the alcohol by drying then results in the formation of a compact membrane exhibiting reduced porosity.

Wicking is an important characteristic for lateral flow kits as it determines the mobility of reagents within the membranes during the washing phase and the time available for performing the immunoassay. Conventional porous membranes tend to have relatively high wicking rates, which prevent the dispensing of aqueous buffers as “wells” in close proximity. This limits the density of “wells” that can be provided on an untreated porous membrane array. By reducing the wicking rate for each reagent droplet dispensed onto the membrane, one would advantageously be able to concentrate the amount of reagent, e.g., capture antibodies, loaded per unit volume.

The disclosed method advantageously allows reagents to be dispensed as discrete spots/wells in close proximity to each other and also with high aspect ratios. More advantageously, the formation of discrete spots/wells can be accomplished without the need for segmentation (including physical or chemical segmentation) of the porous membrane.

Further advantageously, the disclosed method allows for a semi-quantitative analysis of the concentration of a desired analyte, e.g., by impregnating the wells of the porous membrane with varying concentrations of a reagent, e.g., a capture antibody. Semi-quantitative analysis can be performed, e.g., by analyzing the colorimetric intensity of the assay.

In another aspect, there is provided a method for absorbing reagents onto a porous membrane, said method comprising, providing a porous membrane that has been treated with at least one alcohol to reduce its pore size relative to an untreated membrane; dispensing reagents onto said treated porous membrane; absorbing said reagents onto discrete absorption zones on said treated porous membrane.

In some embodiments, the absorption zones may have aspect ratios of from 1:1 to 3:1. In other embodiments, the absorption zones may have aspect ratios of from 2:1 to 3:1.

In still another aspect, there is provided an apparatus for fabricating an array for membrane-based ELISA/p-ELISA, comprising: (a) at least one receptacle housing a porous membrane that has been treated with at least one alcohol; and (b) pressurizing means coupled to said receptacle for generating a localized pressure across said porous membrane.

Advantageously, the above disclosed methods and apparatus are capable of increasing the binding capacity of reagents/probes (e.g. antibodies), which improves the blocking of membranes for superior signal-to-noise ratio in colorimetric ELISA, with comparable results as commercially available ELISA kits.

Further advantageously, the above disclosed methods and apparatus allows for lateral washing of the porous membrane as it is not artificially segmented.

### DEFINITIONS

The following words and terms used herein shall have the meaning indicated:

The word “reagent”, as used in the present specification, refers to a substance that is added to take part in a chemical reaction or to detect the presence of a chemical reaction. In the context of an ELISA, the reagent is typically an antibody, an antigen, an enzyme, or an enzyme conjugated with an antibody and which retains both its enzymatic and immunological activity.

The term “aspect ratio”, as used herein, refers to the ratio between a length feature and a width feature.

When used in respect of an absorption zone/well, it refers to the ratio of the well's depth (which corresponds to the thickness of the porous membrane) and its width feature (e.g. diameter).

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1a shows images of membranes incubated respectively with water, 0.1% Tween, methanol, ethanol and perfluorocarbon liquid (PFCL) and an untreated membrane (as control), stained with luteinizing hormone (LH)-conjugated gold colloid nanoparticles and blue food dye.

FIG. 1b shows images of ethanol-treated membranes dried for varying time periods of 1 hr, 2 hr, 4 hr, 6 hr and 8 hr, respectively, and stained with blue food dye.

FIG. 1c shows scanning electron micrographs (SEMs) of an untreated membrane and an ethanol-treated membrane (dried for 8 hours).

FIG. 1d shows images of the results of a wicking test of an untreated glass fiber membrane as control and an ethanol/aminopropyltrimethoxysilane (APTES)-treated glass fiber membrane.

FIG. 2a is a schematic diagram illustrating one possible arrangement of an apparatus 100 in accordance with the present invention to pattern reagent wells on a porous membrane.

FIG. 2b is a schematic diagram illustrating an alternative arrangement of an apparatus 200 in accordance with the present invention to pattern reagent wells on a porous membrane.

FIG. 2c shows a cross-sectional view of a prototype apparatus 100 in accordance with the illustration of FIG. 2a.

FIG. 2d(i) shows an image of an unwetted membrane screen-printed with Cytop (a hydrophobic, amorphous fluoropolymer) in Example 2.

FIG. 2d(ii) shows an image of the screen-printed membrane after being wetted by a water droplet.

FIG. 2d(iii) shows a cross-section of a porous membrane that has been screen-printed with Cytop and wicking of the water droplet.

FIG. 2e shows images of the top and bottom of an untreated membrane (ii) and an ethanol-treated membrane coated with a laser-cut PTFE hydrophobic film (i) after a wicking test using dyes of different colors. The cross-section of the treated membrane is also shown wherein the diameter of the well is observed to be about 1 mm.

FIG. 2f shows the results of a wicking test performed on treated and untreated Fusion 5™ membranes using a Matrix Equalizer pipette to dispense reagent directly onto the membranes.

FIG. 3a shows an exemplary setup used to enable parallel washing of membranes treated with methanol or ethanol and/or APTES. The setup shows the treated membranes being washed in a 50-mL centrifuge tube via rigorous vortexing.

FIG. 3b shows images of Coomassie-dyed BSA in solvent-treated and solvent/APTES-treated membranes before and after washing in Example 3.

FIG. 3c shows the colorimetric luteinizing hormone (LH) ELISA assay results, using acetone/APTES treated membranes at LH concentrations of 0, 40 and 200 mIU/ml.

FIG. 3d shows the colorimetric LH ELISA assay results from Example 3 using acetone/APTES treated membranes at LH concentrations of 40 and 200 mIU/ml with varying amounts of BSA of ⅛, ¼, ½ and 1 mg/ml.

FIG. 3e shows images of the LH ELISA assay results from Example 3-using Cytop-printed APTES-treated membranes at LH concentrations of 0, 40 and 200 mIU/ml.

FIG. 3f shows images of the LH ELISA assay results from Example 3 using Cytop-printed APTES-treated membranes at LH concentrations of 0, 40 and 200 mIU/ml.

FIG. 3g shows images of, the LH ELISA assay results from Example 3 using Cytop-printed APTES-treated pure glass fiber membranes, with AP and NBT/BCIP being used in the assay.

In the figures, like numerals denote like parts.

### DISCLOSURE OF OPTIONAL EMBODIMENTS

Exemplary embodiments of a method for treating a porous membrane will now be disclosed.

In one aspect, the disclosed method for treating a porous membrane comprises contacting said porous membrane with at least one alcohol to reduce the pore size of said porous membrane relative to an untreated porous membrane.

In certain embodiments, the above method is for preparing a porous membrane having discrete, closely packed data points for use in membrane-based colorimetric ELISA.

Broadly, the porous membrane may be selected from any porous membrane suitable for use as a platform for paper ELISA applications. In some embodiments, the porous membrane may be selected from membranes composed of nitrocellulose, glass fiber, polyvinylidene difluoride (PVDF), dimethylsiloxane (PDMS), filter paper (including but not limited to wood fibers, carbon fibers, quartz fibers), and membranes composed of a mixture of such materials. In one embodiment, the porous membrane is exemplified by a commercially available membrane marketed under the Trademark Fusion 5™ by Whatman (Maidstone, United Kingdom). In another embodiment, the porous membrane is a nitrocellulose membrane. In yet another embodiment, the porous membrane is a glass fiber membrane.

The contacting step may comprise contacting the porous membrane with one or more alcohols having between one to six carbon atoms. In embodiments, the alcohol may be selected from the group consisting of methanol, ethanol, propanol, butanol, pentanol, hexanol, isomers and mixtures thereof. Typical isomers may include, but are not limited to, butan-2-ol, 2-methylpropan-1-ol, 2-methylpropan-2-ol, pentan-2-ol, pentan-3-ol, 2-methylbutan-1-ol, 3-methylbutan-1-ol, 2-methylbutan-3-ol, 2,2-dimethylpropanol, 1-Hexanol, -2-Hexanol, 3-Hexanol, 2-methyl-1-pentanol, 2-methyl-2-pentanol, 2-methyl-3-pentanol, 4-methyl-2-pentanol, 4-methyl-1-pentanol, 3-methyl-1-pentanol, 3-methyl-2-pentanol, 3-methyl-3-pentanol, 2,3-dimethyl-1-butanol, 2,3-dimethyl-2-butanol, 2,2-dimethyl-1-butanol, etc. In one embodiment, the contacting step utilizes a mixture of methanol and ethanol.

The contacting step may further comprise contacting the porous membrane with an organofunctional alkoxysilane compound. The organofunctional alkoxysilane compound may have a general formula, (R¹O)₃—Si—R²X, wherein X represents the organofunctional group and (R¹O) represents an alkoxy group. The organofunctional group X may be selected from amine, epoxide or thiol. Each of R¹ and R² may be independently selected from aliphatic or cyclic, substituted or non-substituted, alkyl, alkenyl, alkyne, aldehyde, or polyolefinic groups. Exemplary organofunctional alkoxysilane compounds include but are not limited to, Methoxy(Polyethyleneoxy)Propyltrimethoxysilane, 7-Octenyltrimethoxysilane, 10-Undecenyltrichlorosilane, 10-Undecenyltrimethoxysilane, 11-(Triethoxysilyl)Udecanal, N-Decyltriethoxysilane, Heptadecafluoro-1,1,2,2-Tetrahydrodecyl)Triethoxysilane, N-Octadecyltrimethoxysilane, N-(Triethoxysilylpropyl)-O-Polyethylene Oxide Urethane, N-Octadecyltrichlorosilane, Triethoxysilylbutyraldehyde, Tetramethyl Orthosilicate, (3-Aminopropyl)Triethoxysilane.

In one embodiment, the organofunctional alkoxysilane comprises an amine functional group, i.e., an aminoalkoxysilane. In yet another embodiment, the organofunctional alkoxysilane compound is aminopropyltrimethoxysilane (APTES).

In certain embodiments, the porous membrane is to be contacted with the alcohol and the organofunctional alkoxysilane compound concurrently.

Advantageously the organofunctional alkoxysilane compound may act as an adhesion promoter for certain types of porous membranes, e.g., glass fiber or PDMS membranes. This can in turn assist in improving the binding of reagents/probes that are to be impregnated on the porous membrane.

The contacting step may further comprise contacting the porous membrane with a solvent mixture comprising at least one or more of the following: water, acetone, a surfactant and a perfluorocarbon liquid (PFCL).

In some embodiments, the contacting step may comprise contacting the porous membrane with one or more alcohols, water, at least one surfactant, at least one PFCL, and an organofunctional alkoxysilane compound concurrently.

Suitable surfactants may include polyoxyethylene (20) sorbitan monolaurate (also termed as “Tween 20”), polyoxyethylene (20) sorbitan monooleate (“Tween 80”), sodium dodecyl sulfate (SDS), polyethylene glycol p-(1,1,3,3-tetramethylbutyl)-phenyl ether (“Triton X-100”), polyoxyethylene monooctylphenyl ether (“Triton X-114”), 3-((3-Cholamidopropyl)dimethylammonium)-1-propanesulfonate (“CHAPS”), Sodium deoxycholate (“DOC”), nonyl phenoxypolyethoxylethanol (“TERGITOL™ NP-40”) and mixtures thereof. Exemplary PFCL compounds may include perfluorohexane.

The contacting step may be performed at room temperature. During the contacting step, the porous membrane may be incubated with a solvent mixture of water, alcohol, surfactant, and PFCL for a duration sufficient to reduce the pore size of the porous membrane relative to an untreated membrane. During the contacting step, the incubating mixture may be subject to physical agitation, e.g., shaking, sonication, etc. In one embodiment, the contacting step may be undertaken for an hour under room temperature conditions which being shaken.

After a sufficient pore size reduction has been obtained, the alcohol may be removed from the porous membrane by drying. The removal of alcohol can be performed by drying via evaporation, under vacuum, by application of heat, e.g., in an oven, or heating under vacuum conditions. The drying step may be undertaken until substantially all the alcohol has been removed. In some embodiments, the porous membrane may be dried for about 1 hour, 2 hours, 3 hours, 4 hours, 5 hours, 6 hours, 7 hours, 8 hours, 9 hours or 10 hours. In one embodiment, the porous membrane was dried from about 4 hours to about 10 hours. In another embodiment, the drying was undertaken for at least 4 hours under vacuum heating.

In certain embodiments, the pore size of a treated porous membrane may be from 20 nm to 100 μm.

Exemplary embodiments of the method for absorbing reagents onto a porous membrane will now be disclosed.

In one embodiment, the method for absorbing reagents onto a porous membrane comprises providing a porous membrane that has been treated with at least one alcohol to reduce its pore size relative to an untreated membrane; dispensing reagents onto said treated porous membrane; absorbing said reagents onto discrete absorption zones on said treated porous membrane.

In certain embodiments, the above method is for preparing a porous membrane having discrete, closely packed, data points for use in membrane-based colorimetric ELISA.

In one embodiment, the porous membrane has been treated in accordance with any one or more method steps already described above. In other embodiments, an untreated porous membrane may be used.

The absorption zones may assume the form of discrete conduits extending through the thickness of the porous membranes (also termed as “wells).

In some embodiments, the average diameters of the absorption zones are from about 0.1 mm to about 1 mm. In certain embodiments, the diameter of the absorption zones may be about 1 mm, or lesser, such as, 0.9 mm or lesser, 0.8 mm or lesser, 0.7 mm or lesser, 0.6 mm or lesser, 0.5 mm or lesser, 0.4 mm or lesser, 0.3 mm or lesser, 0.2 mm or lesser, or 0.1 mm or lesser.

In some embodiments, the absorption zone may have an aspect ratio from 1:1 to 3:1. In some embodiments, the wells have an aspect ratio selected from 1:1, 1.2:1, 1.4:1, 1.6:1, 1.8:1, 2:1, 2.2:1, 2.4:1, 2.6:1, 2.8:1, or 3:1. In one embodiment, the wells have an aspect ratio of at least 2:1.

The absorption zones/wells may be disposed adjacent to each other and are distributed uniformly across the porous membrane to form an array suitable for use in an ELISA. Each absorption zone is spaced distinctly apart and the absorbed contents of each absorption zone do not overlap.

The absorption zones may assume the form of closely packed wells/data points, wherein the spacing between one well to an adjacent well is from 1 to 10 mm. In one embodiment, the spacing between each well is from 1 to 5 mm. Each well may be surrounded by 1 to 8 adjacent wells. The spacing between each well is measured by taking the displacement from the centre of one well to the centre of another well.

The porous membrane may be, of any suitable size to provide an adequate total surface area for preparing a sufficient number of wells. In certain embodiments, the total surface area of the porous membrane may be from 25 to 600 mm², having its length and breadth independently varying from 5 mm to 30 mm. In one embodiment, the porous membrane may be 20 mm by 10 mm in dimension.

Prior to the dispensing step, the disclosed method may further comprise a step of providing a patterned mask on top of the treated porous membrane. The patterned mask may comprise permeable regions permitting passage of reagents through the mask and onto the treated porous membrane. The permeable regions may be distributed substantially uniformly across the patterned mask.

In one embodiment, the patterned mask is a hydrophobic mask comprising permeable regions. In one embodiment, the permeable regions may comprise through-holes. In yet another embodiment, the permeable regions may comprise hydrophilic material which permits the passage of aqueous reagents. The size and distribution of these permeable regions across the mask may be suitably controlled in order to provide an array of discrete wells after the dispensing step.

The size of the permeable regions on the mask may be smaller than the size of the wells formed after the dispensing step. In this regard, the size of the wells may refer to its width, length, diameter or equivalent diameter. In some embodiments, the size of these permeable regions may be from about 0.1 mm to about 5 mm in size. In some embodiments, the permeable regions may be about 4.5 mm, 4 mm, 3.5 mm, 3 mm, 2.5 mm, 2 mm, 1.5 mm, or 1 mm in size. In other embodiments, the permeable regions may be about 0.5 mm, about 0.4 mm, about 0.3 mm, about 0.2 mm, or about 0.1 mm in size.

The hydrophobic mask may be composed of a fluoropolymer. In some embodiments, the mask may be composed of a polytetrafluoroethylene (PTFE) layer or a PTFE tape. The PTFE layer may be provided on the porous membrane via a screen printing step. Alternatively, laser-cut PTFE tape can be used to adhere the mask to the porous membrane. In a preferred embodiment, the hydrophobic mask is provided only on a top surface of the porous membrane. Due to the reduced wicking rate of the treated porous membrane, it is not necessary to provide a hydrophobic layer spanning the entire thickness of the membrane for demarcating discrete wells. Advantageously, this allows lateral washing of the porous membrane, instead of being restricted to just top-down washing.

In one embodiment, the dispensing step may be performed via electronic pipette techniques capable of simultaneously dispensing multiple samples of reagents onto the surface of the porous membrane to form an array of discrete absorption zones. In cases where the pipette technique already provides a sufficiently high resolution of absorption zones, it may be unnecessary to provide a patterned mask prior to the dispensing step. An exemplary, pipette used in such a configuration is the Matrix Equalizer Pipette.

The disclosed method for absorbing reagents on a porous membrane may further comprise providing a localized pressure to increase the flux of reagents through the porous membrane. The localized pressure may be a positive or a negative pressure. A positive pressure may be exerted by the dispensing technique, e.g., when using a pipette. A negative pressure may be applied, for instance, by coupling a vacuum pump to the porous membrane to increase the rate of diffusion of reagents through the membrane. Alternatively, an absorbent layer may be coupled to the porous membrane to increase the flux of reagents through the porous membrane. The absorbent layer may be used independently or in combination with the application of another localized pressure described above. In certain embodiments, the absorbent layer may include porous, absorbent materials with properties similar to polyamide, polycarbonate, polyurethane, polysulfone, polyethersulfone, polyester, cellulose acetate, cellulose nitrate, cellulose triacetate, nitrocellulose and glass fiber.

The disclosed methods may be used to provide a porous membrane having closely packed data points for membrane-based, colorimetric ELISA capable of qualitative and semi-quantitative analysis.

Exemplary embodiments of an apparatus for fabricating an array for p-ELISA will now be disclosed.

In one embodiment, there is provided an apparatus for fabricating an array for p-ELISA, comprising: (a) a receptacle having a porous membrane that has been treated with at least one alcohol disposed therein; and (b) pressurizing means coupled to said receptacle for generating a localized pressure across said porous membrane.

In certain embodiments, the porous membrane is one that has been treated according to any one or more method steps described above.

The receptacle may be substantially planar for receiving the porous membrane therein. The receptacle may comprise through holes to provide fluid communication with a pressurizing means. The receptacle may be arranged such that the pressurizing means exerts a substantially uniform localized pressure across the entire porous membrane when housed within the receptacle. The receptacle may further comprise securing means for securing the porous membrane in position. In one embodiment, the securing means may act to hold the porous membrane flat across the planar surface of the receptacle such that a uniform pressure can be exerted across substantially the entire surface of the porous membrane. In one embodiment, the securing means may be a slit, adapted to engage at least an edge of the porous membrane, to thereby secure the membrane in place. The securing means may comprise two or more slits, each slit being adapted to receive and engage an edge of the porous membrane.

The apparatus may further comprise a patterned mask which is provided on top of the porous membrane. The patterned mask may be one that is as described above. In one embodiment, the patterned mask may comprise permeable regions which permit passage of reagents through the mark and onto the porous membrane. The apparatus may further comprise at least one absorbent layer that is coupled to the porous membrane. Advantageously, the absorbent layer may act to increase the rate of diffusion of reagents across the porous membrane.

In some embodiments, the through holes provided on the receptacle to establish fluid communication with the pressurizing means may be substantially aligned with the permeable regions of the hydrophobic mask. Advantageously, such a configuration is able to enhance the absorption rate of the reagent through the membrane. More advantageously, such a configuration reduces wicking of the reagent when being transported through the membrane, resulting in wells that have small radii.

The disclosed apparatus may be used to provide a porous membrane having closely packed data points for membrane-based colorimetric ELISA capable, of qualitative and semi-quantitative analysis.

### EXAMPLES

Non-limiting examples of the invention will be further, described in greater detail by reference to specific Examples, which should not be construed as in any way limiting the scope of the invention.

Materials

The materials used in the following examples are listed below.

The Fusion 5™ membrane was purchased from Whatman (Maidstone, UK).

Aminopropyltrimethoxysilane (APTES), bovine serum albumin (BSA), phosphate buffered saline (PBS), methanol, ethanol, Coomassie dye, Tween® 20, horseradish peroxidase (HRP) and 3,3,5,5-tetramethylbenzidine (TMB), alkaline phosphatase (AP) were purchased from Sigma Aldrich (St. Louis, Mo., USA), and perfluorocarbon liquid (PFCL) and polytetrafluoroethylene (PTFE) film tapes were obtained from 3M (St. Paul, Minn., USA).

Cytop (a hydrophobic, amorphous fluoropolymer) was obtained from AGC Chemicals Europe, Ltd (Lancashire, UK).

The luteinizing hormone (LH) ELISA kit was from DRG International (Mountainside, N.J., USA). The monoclonal anti-LH gold conjugate was from Arista Biologicals (Allentown, Pa., USA) and the anti-LH-AP conjugate was formed using the Lightning-Link™ Labeling Kit from Innova Biosciences Ltd (Cambridge, UK).

Imaging, Screen Printing and Prototype Development

The equipment used in the following examples are described below.

The scanning electron microscopy (SEM) images were obtained using Jeol JSM7400F (Tokyo, Japan).

Other images were obtained with Panasonic Lumix DMC-FS2 (Tokyo, Japan) or Leica MZ16 FA stereomicroscope (Wetzlar, Germany) at 10× magnification.

Screen printing was done using the Hanky Mid-size Flat Screen Printer TP-400FS (Taipei, Taiwan).

The prototypes developed were designed by Solidworks (Waltham, Mass., USA), and prototyped using. Objet Eden 350 (Billerica, Mass., USA).

Matrix Equalizer pipettes were obtained from Thermo Fisher Scientific (MA, USA).

### Example 1

Example 1 investigates the properties of a porous membrane that has been treated in accordance with one or more methods according to the present disclosure.

A Fusion 5™ porous membrane was incubated, respectively, with H₂O, 0.1% Tween® 20 (a surfactant), methanol, ethanol and PFCL for 1 hr on a shaker before drying under vacuum for 2 hr.

After treatment, the wicking properties of the various treated membranes were compared with an untreated membrane serving as a control. The comparison was done using luteinizing hormone (LH)-conjugated gold nanoparticles having a concentration of 1 mg/ml and blue food dye as the absorbent reagent.

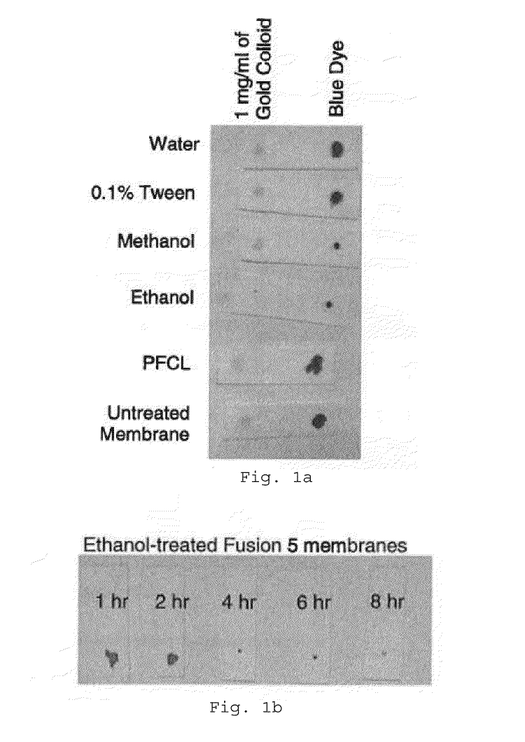

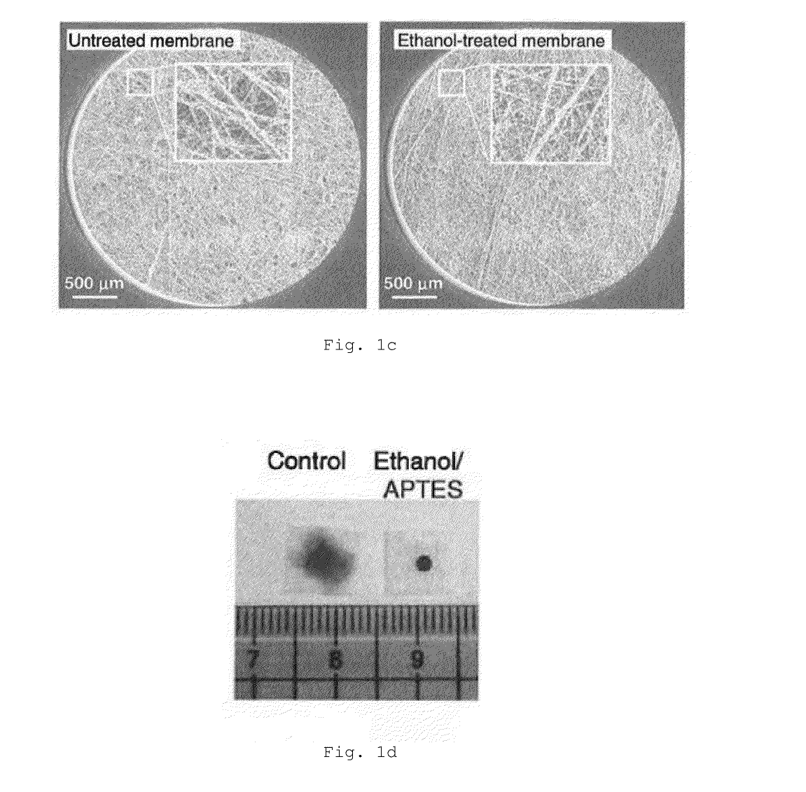

2 μl of LH-conjugated gold nanoparticles and 2 μl of blue food dye were dispensed onto, the membranes and imaged after 1 min. The results are shown in FIG. 1a.

It is evident from the relatively small, well-defined spots on the ethanol- and methanol-treated membranes that the wicking rates of these membranes were reduced significantly as compared to membranes treated with other compounds and the untreated membrane. It was observed that the dispensed reagent would eventually wick even in an ethanol-treated membrane, albeit with a relatively smaller wicking radius. One possible explanation that may be inferred from these results is that the ethanol- and methanol-treatment increased hydrophobicity.

The effect of drying time was investigated using an ethanol-treated membrane. 2 μl of blue food dye was dispensed onto the ethanol-treated membranes dried for time periods of 1 hr, 2 hr, 4 hr, 6 hr and 8 hr, respectively. The results are shown in FIG. 1b. The results suggest that wicking rate can be reduced with drying time of 1 hour or above. The results further suggest that optimal results may be reached with drying time of at least 4 hours.

Scanning electron micrographs (SEMs) of an untreated membrane and an ethanol-treated membrane (dried for 8 hours) were taken. The SEMs are shown in FIG. 1c. The contrasting micrographs show that the ethanol treated membrane has a more compact microstructure compared to the untreated membrane. In particular, a significant pore size reduction can be observed after the treatment.

The effect of alcohol treatment on wicking rate was also investigated on a hydrophilic glass fiber membrane (obtained from Whatman). The glass fiber membrane was incubated overnight with ethanol and an aminoalkoxysilane compound (APTES) and heated at 100° C. for 2 hours. The results of a wicking test (with 2 μL blue dye) performed on a control membrane and an ethanol/APTES treated membrane are shown in FIG. 1d. Again, it can be observed that the treated membrane demonstrates a controlled wicking rate with the absorbed reagent forming a well-defined, discrete spot having a small radius.

### Example 2

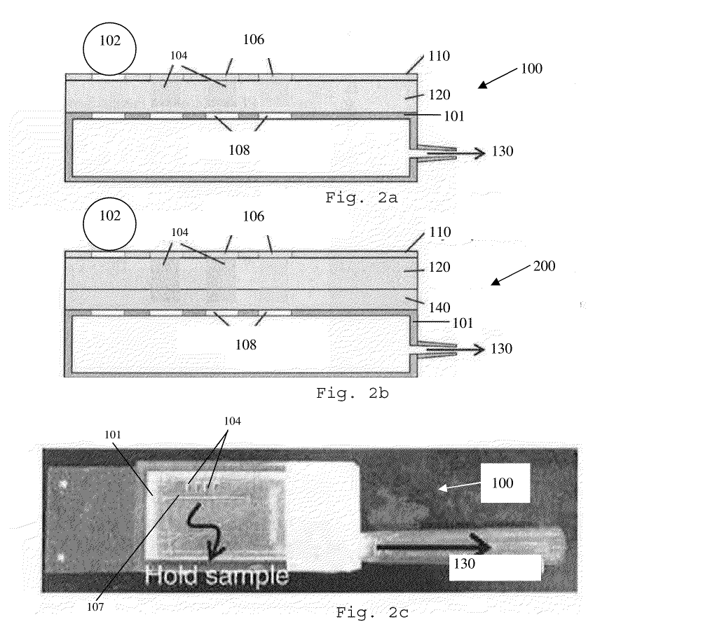

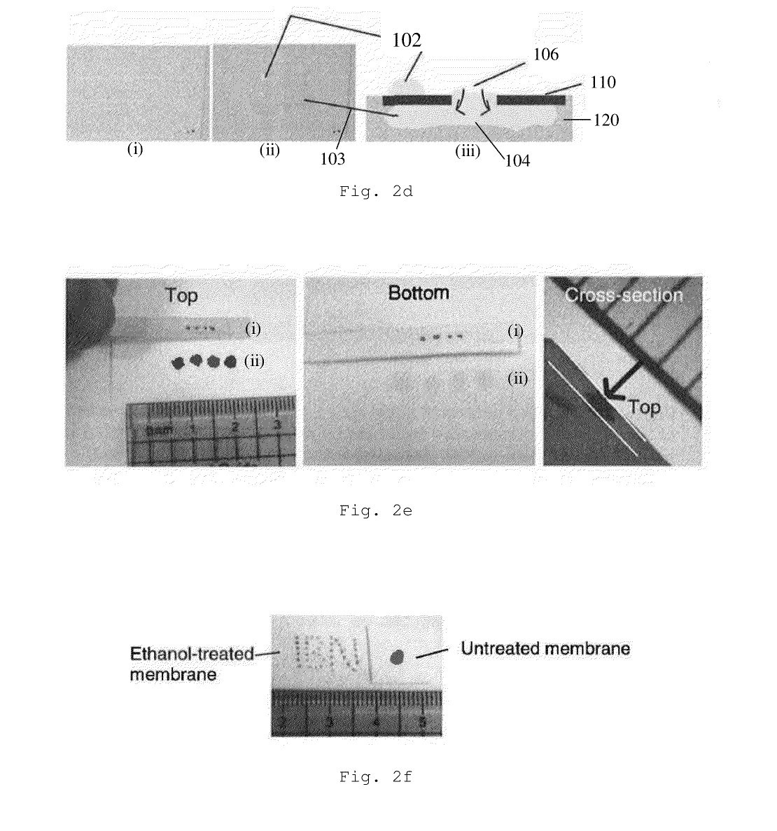

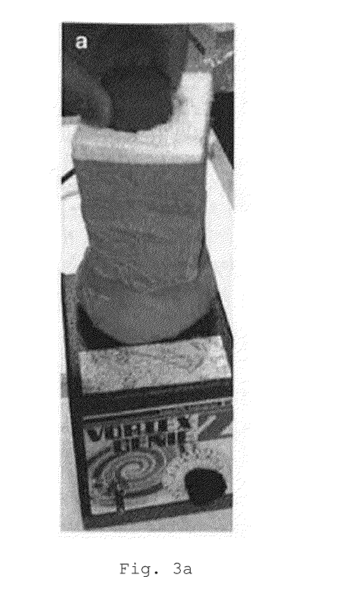

In this example, a strategy was devised to pattern wells in high resolution on treated and untreated porous membranes. Two approaches can be used to pattern wells on, the membranes. FIG. 2a depicts an exemplary apparatus 100 for this purpose, while another exemplary apparatus 200 is illustrated in FIG. 2b. Both apparatus arrangements 100 and 200 employ a localized pressure difference to deposit aqueous reagents within and through the porous membranes. A cross-sectional view of a prototype apparatus 100 as described above in the first approach is shown in FIG. 2d. The apparatus 100 contains a receptacle 101 for receiving a porous membrane 120. A horizontal slit 107 is provided within the receptacle 101 for engaging and securing the porous membrane 120 being placed therein. In particular, the slit 107 ensures that the porous membrane 120 is held flat uniformly on the apparatus due to pressure difference. The receptacle 101 contains through holes 108 which are fluidly communicated with a vacuum source 130, for applying a localized pressure across the membrane 120.

Referring to FIGS. 2a and 2b, a hydrophobic mask 110 is provided on the surface of a porous membrane 120 to demarcate the areas for well formation. The hydrophobic mask 110 contains permeable regions 106, which can be hydrophilic features or through holes, and which permit an aqueous reagent 102 to pass through. The aqueous reagent 102 is then absorbed on the treated or untreated porous membrane 120 beneath the mask 110, forming reagent “wells” 104. In this manner, a plurality of reagent wells 104 may be patterned on the porous membrane 120. The absorption of reagents may be assisted by the application of a localized pressure by a vacuum 130. It can be noted that the through holes 108 are aligned with the permeable regions 106.

The second embodiment shown in FIG. 2b differs from the arrangement in FIG. 2a in that an absorbent membrane 140 is provided beneath the porous membrane 120, such that the porous membrane 120 is sandwiched between the mask 110 and the absorbent layer 140. It can also be seen that the through holes 108 of the receptacle 101 are substantially aligned with the through holes 106 on the hydrophobic mask 110.

FIG. 2(d)(iii) shows a cross-section of a porous membrane 120 that has been screen-printed with a hydrophobic layer 110 (Cytop, which is a hydrophobic, amorphous fluoropolymer) having a through hole 106 for an aqueous reagent to pass through in the direction of the arrows shown and absorb onto the porous membrane 120. The screen printing of a hydrophobic layer 110 allows the spots/wells 104 to be demarcated for easy visual identification and analysis. Once absorbed onto the porous membrane 120, the aqueous reagent 102 may wick laterally across the porous membrane, forming a wetted area 103 that may be larger than the size of the through hole. To illustrate the presence of the hydrophobic layer on the unwetted membrane shown in FIG. 2d(i), the membrane was wetted with a water droplet and the image is shown in FIG. 2d(ii).

The use of a treated and untreated Fusion 5™ membrane, each coated with a hydrophobic layer for patterning spots in high resolution, is next illustrated.

A piece of hydrophobic laser-cut PTFE film was placed onto an ethanol-treated membrane as described above. A film was used in this example to easily identify the through holes that were aligned with the device for spotting purposes.

The membrane with the hydrophobic film was then aligned with the holes on the device as illustrated in FIG. 2c. The vacuum was activated before dispensing 1 μL of dyes of different colors and the resulting top, bottom and cross-sectional images are shown in FIG. 2e.

Without treatment of the membrane, the 1 μL dye drops wicked significantly in a lateral manner as shown from the top and bottom of the untreated membrane in FIG. 2e(ii).

Conversely, using the combined approach of ethanol treatment and hydrophobic coating, distinct dye drops are observed from the top and bottom of the ethanol-treated membrane in FIG. 2e(i). As can be seen from FIG. 2e(i), the colored dye permeated the entire porous membrane to form well-defined patterns with an aspect ratio (depth:width) of 1:0.5.

Another alternative to pattern spots in high resolution onto a treated and untreated Fusion 5™ membrane, each coated with a hydrophobic layer, is to use the Matrix Equalizer pipette to dispense directly onto the membrane.

An image obtained using this alternative method is shown in FIG. 2f. As seen from FIG. 2f, spots in close proximity were patterned due to the positive pressure generated from dispensing process as compared to an untreated membrane.

### Example 3

The effects of aminopropyltrimethoxysilane (APTES) on porous membranes for protein immobilization were investigated in this example. Solvent-treated and solvent/APTES-treated Fusion 5™ membranes in accordance with Example 1 were used in this example. The solvents used were methanol, ethanol and acetone.

Methanol or Ethanol APTES-Treated Membranes

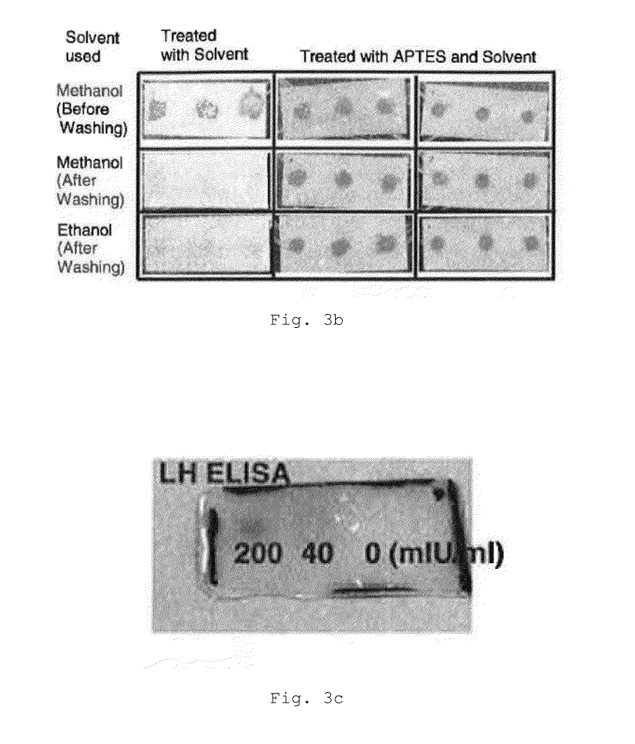

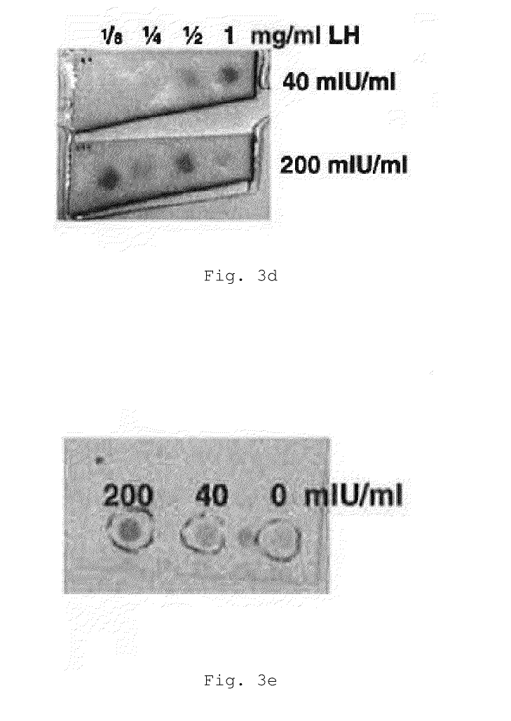

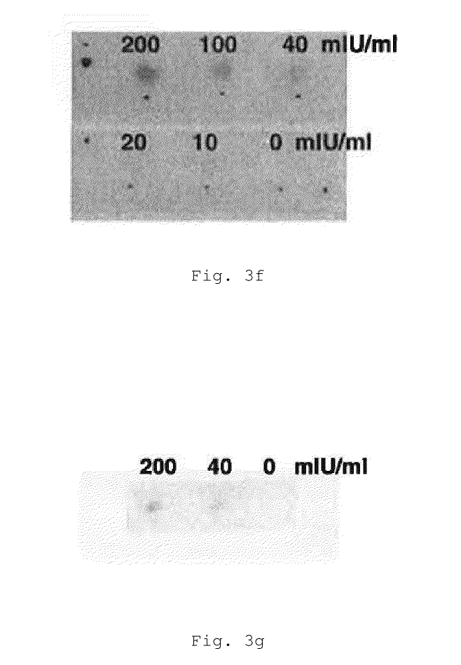

The solvents used here were methanol and ethanol. 1% bovine serum albumin (BSA) (dissolved in deionized H₂O) as the capture antibody was dispensed onto solvent treated and solvent/APTES-treated Fusion 5™ membranes, which were then washed in a 50-mL centrifuge tube via rigorous vortexing. The setup used to enable parallel washing of the treated membranes is shown in FIG. 3a.

Coomassie dye was used as staining to indicate the presence of any BSA remaining on the membranes. The stained images of solvent-treated membranes before and after washing, as well as solvent/APTES-treated membranes before and after washing, are shown in FIG. 3b. In FIG. 3b, it can be seen that the solvent/APTES-treated Fusion 5™ membrane was found to retain BSA within the membrane as evidenced by the pronounced dots. This is important when the membrane is used for ELISA assays because BSA is commonly used as a blocking agent, i.e. capture antibody.

Acetone/APTES-Treated Membranes

Fusion 5™ membranes treated with APTES and acetone were used here to conduct ELISA at three concentrations of luteinizing hormone (LH), i.e. 0, 40 and 200 mIU/ml.

The reagents used here have been validated in a standard 96-well plate ELISA. In translating the 96-well plate ELISA to paper ELISA as used in this example, the various conditions such as reagent and incubation time used were not altered. Only the concentration of the analyte (LH) was varied as described above. The enzyme and substrate used in the ELISA assay here were horseradish peroxidase (HRP) and 3,3,5,5-tetramethylbenzidine (TMB).

The colorimetric LH ELISA results are shown in FIG. 3c. It can be seen from FIG. 3c that the samples containing 40 and 200 mIU/ml of LH showed distinctive colored spots. Negligible colorimetric signal was observed in the negative control, i.e. 0 mIU/ml of LH.

This demonstrated the successful conversion of ELISA conducted on a 96-well plate to ELISA on a porous membrane (i.e. paper ELISA), without compromising the detection limit and signal-to-noise ratio of the assay. When acetone was employed as a solvent for APTES to treat a porous membrane, the hydrophilicity of the membrane was retained to thereby control the wicking of the treated membrane designed for ELISA.

The effectiveness of the treated membrane in accordance with one or more methods according to the present disclosure when used in an ELISA assay was investigated using an acetone/APTES-treated Fusion 5™ membrane. The amounts of capture antibodies (BSA) on the treated membrane were varied at ⅛, ¼, ½ and 1 mg/ml for LH concentrations of 40 mIU/ml and 200 mIU/ml, respectively.

With this method, the concentrations of LH can, directly be estimated by the number and intensity of blue spots observed, without the need for a standard calibration curve. The results of the ELISA assays are shown in FIG. 3d.

In FIG. 3d, it can be seen that at 200 mIU/ml of LH, dark-colored spots can be seen for all four amounts of BSA. At 40 mIU/ml of LH, only two dark-colored spots can be seen at the higher amounts of ½ and 1 mg/ml of, BSA, while one light-colored spot can be seen at ¼ mg/ml of BSA. No spots could be seen at ⅛ mg/ml of BSA for the 40 mIU/ml LH sample.

Patterned Membranes in ELISA

The effectiveness of membranes patterned in accordance with the methodology described in Example 2 and used in an ELISA assay was investigated. Further, membranes patterned in accordance with the methodology described in Example 2 were incorporated into ELISA. Specifically, Cytop-printed APTES-treated Fusion 0.5™ membranes were incorporated into an LH ELISA assay.

Again, three concentrations of LH, i.e. 0, 40 and 200 mIU/ml, were used to conduct ELISA. The results of the assay (FIG. 3e) show that Cytop-printed treated membranes are able to give clear, distinct demarcations of the data points. Specifically, in FIG. 3e, clear distinct circular colored spots for 200 and 40 mIU/ml of LH can be visually observed. This indicates the feasibility of utilizing the disclosed patterning methodology for membranes used in ELISA assays.

The sensitivity of the ELISA assay was further investigated by changing the enzyme and substrate from HRP and TMB to alkaline phosphatase (AP) and nitro-blue tetrazolium/5-bromo-4-chloro-3′-indolyphosphate (NBT/BCIP), respectively. The ELISA was conducted again with AP and NBT/BCIP and the assay results are shown in FIG. 3f.

The colorimetric ELISA results (FIG. 3f) shows increased signal intensity as the LH concentration was increased from 0 to 200 mIU/ml, demonstrating the feasibility of the disclosed methods and membranes for use with different ELISA reagents.

The effectiveness of patterned and treated glass fiber membranes used in ELISA assays was also investigated. LH-ELISA was conducted again with AP and NBT/BCIP using Cytop-printed APTES-treated pure glass fiber membrane. The assay results (FIG. 3g) demonstrate that the APTES modification technique can also be applied on pure glass fiber membranes and incorporated into ELISA.

### APPLICATIONS

The presently disclosed method for treating a porous membrane is expected to see utility in a variety of diagnostic kits, e.g., pregnancy test kits, and other analyte-detection applications.

The reduced wicking rate of the treated membrane allows the porous membrane to be advantageously used as a platform for forming densely distributed but well-demarcated reagent wells for performing an assay. Further advantageously, the formation of these wells can be accomplished without the need for segmentation of the porous membrane, e.g., via introduction of physical barriers extending through the thickness of the membrane. Using the treated membrane as a starting platform, the disclosed apparatus further builds upon the technical benefits of the treated porous membrane for fabricating an assay intended for use in a paper-ELISA.

It will be apparent that various other modifications and adaptations of the invention will be apparent to the person skilled in the art after reading the foregoing disclosure without departing from the scope of the invention and it is intended that all such modifications and adaptations come within the scope of the appended claims.

1. A method for reducing wicking rate in a porous membrane, said method comprising contacting said porous membrane with at least one alcohol to reduce the pore size of said porous membrane relative to an untreated porous membrane.

2.-23. (canceled)

24. The method of claim 1, wherein the pore size of a treated porous membrane is from 20 nm to 100 μm.

25. The method of claim 1, wherein said contacting step comprises contacting porous membrane with alcohols having between one to six carbon atoms, said alcohol selected from the group consisting of: methanol, ethanol, propanol, butanol, pentanol, hexanol and mixtures thereof.

26. The method of claim 1, wherein said contacting step further comprises contacting said porous membrane with an organofunctional alkoxysilane comprising at least one functional group selected from amine, epoxide or thiol.

27. The method of claim 26, wherein said contacting step further comprises contacting said porous membrane with a solvent mixture containing at least one or more of water, a surfactant and a perfluorocarbon.

28. The method of claim 1, further comprising drying the porous membrane after the contacting step.

29. A method for absorbing reagents onto a porous membrane, said method comprising,
providing a porous membrane that has been treated with at least one alcohol to reduce its pore size relative to an untreated membrane;
dispensing reagents onto said treated porous membrane; and
absorbing said reagents onto discrete absorption zones on said treated porous membrane.

30. The method of claim 29, wherein each absorption zone has an aspect ratio of from 1:1 to 3:1.

31. The method of claim 30, wherein the spacing between the centre of one absorption zone to the centre of an adjacent absorption zone is from about 1 mm to about 10 mm and wherein the diameter of each absorption zone is from about 0.1 mm to about 1 mm.

32. The method of claim 29, further comprising, prior to said dispensing step, a step of providing a patterned mask on said treated porous membrane, wherein said patterned mask comprises permeable regions permitting passage of said reagents onto the treated porous membrane.

33. The method of claim 32, wherein said patterned mask is substantially hydrophobic.

34. The method of claim 29, further comprising providing a localized pressure to increase the flux of reagents through the porous membrane.

35. The method of claim 34, wherein a negative pressure is being applied.

36. The method of claim 1, for preparing a porous membrane having closely packed data points for membrane-based, colorimetric ELISA that is capable of qualitative and semi-quantitative analysis.

37. The method of claim 29, wherein an untreated porous membrane is used in place of a treated membrane.

38. A diagnostic kit comprising a treated porous membrane prepared according to claim 1.

39. An apparatus for fabricating an array, comprising:
(a) a receptacle having a porous membrane that has been treated with at least one alcohol disposed therein, the porous membrane having a reduced wicking rate relative to an untreated membrane; and
(b) pressurizing means coupled to said receptacle for generating a localized pressure across said porous membrane.

40. The apparatus of claim 39, further comprising a patterned mask disposed on top of said porous membrane, said patterned mask comprising permeable regions which permit passage of aqueous reagents through the mask and onto said porous membrane.

41. The apparatus of claim 40, wherein said receptacle comprises through holes which are fluidly communicated with said pressurizing means.

42. The apparatus of claim 41, wherein said through holes are substantially aligned with said permeable regions of said patterned mask.

43. An apparatus for fabricating an array, comprising:
(a) a receptacle having a porous membrane that has been treated with at least one alcohol disposed therein, the porous membrane having a reduced wicking rate relative to an untreated membrane; and
(b) pressurizing means coupled to said receptacle for generating a localized pressure across said porous membrane.

44. The apparatus of claim 43, further comprising a patterned mask disposed on top of said porous membrane, said patterned mask comprising permeable regions which permit passage of aqueous reagents through the mask and onto said porous membrane.

45. The apparatus of claim 44, wherein said receptacle comprises through holes which are fluidly communicated with said pressurizing means.

46. The apparatus of claim 45, wherein said through holes are substantially aligned with said permeable regions of said patterned mask.

47. A porous membrane prepared according to claim 1.
